(19) United States
(12) Patent Application Publication
(10) Pub. No.: US 2005/0209201 A1
(43) Pub. Date: Sep. 22, 2005

(54) Method and composition for suppression of inflammation

(75) Inventors: Kaori Inoue, Yokohama-shi (JP); Mitsuhiro Denda, Yokohama-shi (JP); Shigeyoshi Fujiwara, Yokohama-shi (JP)

Correspondence Address: SNIDER & ASSOCIATES, P. O. BOX 27613, WASHINGTON, DC, 20038-7613, US

(73) Assignee: Shiseido Co., Ltd., Tokyo (JP)

(21) Appl. No.: 11/077,389

(22) Filed: Mar. 11, 2005

(30) Foreign Application Priority Data
Mar. 16, 2004 (JP) 2004-074638

(51) Int. Cl. A61K 31/655

(52) U.S. Cl. 514/150

(57) ABSTRACT

The present invention provides a method and composition which ameliorate inflammation, and suppress its onset, by allowing ATP receptor antagonists to act on ATP receptors of cells to block them and thereby inhibit the release of inflammatory cytokines, particularly interleukin-6 (IL-6) and/or interleukin-8 (IL-8), by the cells.

### FIELD OF THE INVENTION

The present invention relates to a method and composition which ameliorate inflammation and suppress its onset by allowing an ATP receptor antagonist to act on ATP receptors of cells to block them and thereby inhibit release of inflammatory cytokines, especially interleukin-6 (IL-6), interleukin-8 (IL-8), interleukin-1α (IL-1α) and/or tumor necrosis factor α (TNFα), by these cells.

### PRIOR ART

External stimuli such as ultraviolet irradiation, dryness and exposure to chemical agents inflict various sorts of damage to skin. In particular, skin redness accompanying ultraviolet-induced inflammation (sunburn) not only produces pain and a burning sensation, but in severe cases can cause blistering similar to that induced by heat injury.

One of the causes of inflammation induced by different external stimuli such as ultraviolet rays is the production of active oxygen or free radicals. Generation of active oxygen or free radicals in the skin damages cells, creating “sunburn cells” and producing genetic damage (DNA damage), and accumulated DNA damage over prolonged periods can even lead to skin cancer. Pharmaceutical agents, natural remedies and antioxidants are therefore commonly used to suppress skin inflammation. In addition, steroids with anti-inflammatory effects, various humectants or animal and vegetable extracts which exhibit moisture retentive effects are also used to attenuate inflammation.

### DISCLOSURE OF THE INVENTION

The object of the present invention is to provide suppression and amelioration of cutaneous inflammation, induced by external stimuli such as ultraviolet irradiation, by an approach which is absolutely new and different from the conventional anti-inflammatory mechanisms.

As is well known, ATP is released from cells subjected to injury by mechanical stimuli such as tape stripping, or by other cell-damaging external stimuli, and the released ATP binds to ATP receptors to initiate signal transduction (Pain 95(2002)41-47, S. P. Cook and E. W. McClesky; J. Invest. Dermatol. 119(2002)1034-1040, M. Denda, K. Inoue, S. Fuziwara, S. Denda). However, no published reports exist from research focusing on and elucidating the relationship between released ATP and inflammation. The present inventors have discovered, surprisingly, that the inflammatory cytokines IL-6, IL-8, TNF and IL-1α are released by the action of ATP on keratinocytes. They have also discovered that release of these cytokines can be inhibited by blocking ATP receptors on keratinocytes. While ATP is known to be involved with in vivo energy metabolism and signal transduction, the fact of its role in inflammation has been completely unknown in the prior art, and is a surprising discovery.

According to a first aspect, therefore, the present invention provides a method for suppression of inflammation characterized by allowing an ATP receptor antagonist to act on ATP receptors of cells to block the receptors, thereby inhibiting release of inflammatory cytokines by the cells. The ATP receptors are preferably cell membrane receptors.

According to a second aspect, the invention provides a pharmaceutical composition or cosmetic composition for suppression of inflammation, characterized by comprising an ATP receptor antagonist in an amount effective to act on ATP receptors of cells, thereby inhibiting release of inflammatory cytokines by the cells.

According to the invention it is possible to treat and ameliorate external stimulus-induced inflammation by an absolutely new and effective means, without side effects, which does not exist in the prior art.

### BRIEF DESCRIPTION OF THE DRAWINGS

FIG. 1 shows the results of assaying ATP-induced IL-6 release from keratinocytes and examining the inhibitory effect of Reactive blue 2 on IL-6 release, using ELISA. The horizontal axis represents ATP concentration (μM), and the vertical axis represents culture supernatant IL-6 concentration (μg/ml).

FIG. 2 shows the results of assaying ATP-induced IL-8 release from keratinocytes and the examination of the inhibitory effect of Reactive blue 2 on IL-8 release, using ELISA. The horizontal axis represents ATP concentration (μM), and the vertical axis represents culture supernatant IL-8 concentration (μg/ml).

FIG. 3 shows the results of assaying ATP-induced IL-1α release from keratinocytes and the examination of the inhibitory effect of Reactive blue 2 on IL-1α release, using ELISA. The horizontal axis represents ATP concentration (μM), and the vertical axis represents culture supernatant IL-1α concentration (μg/ml).

FIG. 4 shows the results of assaying ATP-induced TNFa release from keratinocytes and the examination of the inhibitory effect of Reactive blue 2 on TNFα release, using ELISA. The horizontal axis represents ATP concentration (μM), and the vertical axis represents culture supernatant TNFα concentration (μg/ml).

FIG. 5 shows the effect of ATP addition to cellular IL-6 gene expression by PCR. The horizontal axis represents ATP concentration (μM), and the vertical axis represents culture supernatant IL-6 gene expression, normalized on the basis of GAPDH gene expression.

FIG. 6 shows the effect of ATP receptor antagonist treatment on IL-6 gene expression by PCR. The vertical axis represents culture supernatant IL-6 gene expression, normalized on the basis of GAPDH gene expression.

FIG. 7 shows the effect of ATP addition to cellular IL-8 gene expression by PCR. The horizontal axis represents ATP concentration (μM), and the vertical axis represents culture supernatant IL-8 gene expression, normalized on the basis of GAPDH gene expression.

FIG. 8 shows the effect of ATP receptor antagonist treatment on IL-8 gene expression by PCR. The vertical axis represents culture supernatant IL-8 gene expression, normalized on the basis of GAPDH gene expression.

FIG. 9 shows the result of an experiment confirming release of ATP from keratinocytes by ultraviolet stimulation. The horizontal axis represents ultraviolet ray (UVB) intensity (mJ/cm²), and the vertical axis represents culture supernatant ATP concentration (nM).

FIG. 10 shows the effect of an ATP antagonist on cytokine IL-6 release after ultraviolet irradiation. The vertical axis represents culture supernatant IL-6 concentration (μg/ml).

FIG. 11 shows the effect of an ATP antagonist on cytokine IL-8 release after ultraviolet irradiation. The vertical axis represents culture supernatant IL-8 concentration (μg/ml).

FIG. 12 shows skin histology by HE staining, with water coating (upper panel) and 1 mM Reactive blue 2 coating (lower panel), using acetone-treated HR-1 mice in a dry environment (<10% RH).

### BEST MODE FOR CARRYING OUT THE INVENTION

As explained above, the present invention provides a method and a composition which suppresses the onset of inflammation. By allowing ATP receptor antagonists to block ATP receptors, the release of inflammatory cytokines will be inhibited. The ATP receptors referred to here are those present on the surfaces of mammalian cells, and preferably on cutaneous cells such as keratinocytes, which compose the horny layer, cuticle, basal membrane and dermis.

ATP (adenosine triphosphate) is synthesized in the mitochondria of all cells and is used as an energy source for biological reactions, but it is also known to function as an intercellular signal transduction molecule. ATP-binding receptors were cloned in 1993 and, based on their features, they have been generally categorized as either gated ion-channel receptor types (P2Xn) or G protein-coupled receptor types (P2Yn). Currently, P2Xn includes 7 different subtypes while P2Yn includes 9 different subtypes. There are two receptor types in human keratinocytes, P2Y and P2X. P2Y receptors include four subtypes, P2Y1, P2Y2, P2Y4 and P2Y6. P2X receptors include two subtypes, P2X5 and P2×7. The P2X3 receptor subtype is expressed only in mice. (References: Greig A V H, Linge C, Terenghi G, McGrouther, Burnstock G. Purinergic receptors are part of a functional signaling system for proliferation and differentiation of human epidermal keratinocytes. J Invest Dermatol 2003: 120: 1007-1015; Dixon C J, Bowler W B, Littlewood-Evans A, Dillon J, Bilbe G, Sharpe G R, Gallagher. Regulation of epidermal homeostasis through P2Y₂ receptors. Br J Pharmacol 1991: 127: 1680-1686; Denda M, Inoue K, Fuziwara S, Denda S. P2X purinergic receptor antagonist accelerates skin barrier repair and prevents epidermal hyperplasia induced by skin barrier disruption. J Invest Dermatol 2002: 119: 1034-1040; Burrell H E, Bowler W B, Gallagher J A, Sharpe G R. Human keratinocytes express multiple P2Y-receptors: Evidence for functional P2Y1, P2Y2, and P2Y4 receptors. J Invest Dermatol 2003: 120: 440-447). The mammals referred to here include not only humans but also other species such as monkeys, dogs, cats, mice, rats, rabbits, horses, cows, sheep and goats.

There are various natural and synthetic compounds such as Reactive blue (anthraquinone-sulfonic acid derivative) 2, suramin, PPADS (pyridoxalphosphate-6-azophenyl 2′,4′-disulfonic acid), TNP-ATP (trinitrophenyl-ATP), Brilliant Blue G, IP₅I that antagonize ATP receptors. A particularly effective ATP receptor antagonist for the invention is Reactive blue 2.

The inflammatory cytokines that are suppressed by the method and composition of the present invention are IL-6 and IL-8. These cytokines are known to be released from cells upon binding of ATP to ATP receptor on the cells to produce inflammation. Examples of other inflammatory cytokines include interleukin-1α,β, interleukin-18, granulocyte/macrophage-colony stimulating factor (GM-CSF) and tumor necrosis factor (TNF).

The term “inflammation” used according to the present invention refers to skin inflammation caused by stress, and especially external stimuli such as ultraviolet irradiation, dry irritation, heat irritation (hot and cold), chemical agent irritation, osmotic irritation, oxidative irritation. More preferably, the inflammation is skin inflammation induced by ultraviolet irradiation or dry irritation.

The method of ameliorating or suppressing onset of inflammation according to the present invention may carried out by applying to a site of inflammation a composition comprising an ATP receptor antagonist, and preferably Reactive blue 2. The composition may be applied to the site of inflammation as a pharmaceutical composition, or as a cosmetic, in the form of an external application, for example.

The pharmaceutical or cosmetic composition of the present invention will normally be prepared by adding the ATP receptor antagonist, preferably Reactive blue 2, to an aqueous solvent such as water or ethanol. The content of the ATP receptor antagonist is not particularly restricted according to the invention, and for example, the solution used may have a concentration in a range of 1 μM to 10 mM, preferably 10 μM to 1 mM and more preferably about 100 μM. When the agent of the present invention is prepared as a bath/shower cosmetic, it will usually be diluted to about 100- to 1000-fold at the time of use, and it is preferably prepared at a high concentration with this in mind. As aqueous solvents, lower alcohols are more suitable, where the lower alcohol content of the composition is preferably 20-80 wt % and more preferably 40-60 wt %.

In addition to the ATP antagonist as the essential component, the pharmaceutical or cosmetic composition of the present invention may also contain other components ordinarily used in cosmetic or pharmaceutical external applications, such as whiteners, humectants, antioxidants, oil components, ultraviolet absorbers, surfactants, thickeners, alcohols, powder components, pigments, aqueous components, water or various skin nutrients, as necessary and appropriate.

Depending on the purpose of the composition, it may also contain, in appropriate amounts, metal sequestering agents such as disodium edetate, trisodium edetate, sodium citrate, sodium polyphosphate, sodium metaphosphate and gluconic acid, drug agents such as caffeine, tannin, verapamil, tranexamic acid and its derivatives, licorice extract, glabridin, quince fruit hot water extract, various galenicals, tocopherol acetate, and glycyrrhizinic acid and its derivatives or salts, other whiteners such as vitamin C, magnesium ascorbate phosphate, ascorbic acid glucoside, albutin and kojic acid, sugars such as glucose, fructose, mannose, sucrose and trehalose, and vitamin A derivatives such as retinoic acid, retinol, retinol acetate and retinol palmitate.

A pharmaceutical or cosmetic composition of the present invention may be an external preparation for cosmetic, pharmaceutical or medical use, in the form of any conventional external skin formulation such as, for example, cosmetic water, cream, an emulsion, lotion, pack, bath/shower agent, ointment, hair lotion, hair tonic, hair liquid, shampoo, rinse, hair growth tonic or the like, depending on the purpose of use, with no particular restrictions on the type of formulation.

### EXAMPLES

The present invention will now be explained in greater detail by the following examples.

Materials and Methods

(1) Culturing of Normal Human Keratinocytes

Commercially available keratinocytes (Kurabo) were cultured in KGM-2 medium (Kurabo) according to the manufacturer's manual. The cells were seeded in a 12-well plate at 1.5×10⁵ cells/well.

(2) Assay of Cytokines (IL-6, IL-8, IL-1α and TNF) in Culture Supernatants

The medium in the 12-well plate was discarded on the day after seeding of the cells, and 24 hours after adding 2 ml of test solution dissolved in fresh medium, the culture supernatants were collected. The IL-6, IL-8, IL-1α and TNF concentrations in the recovered supernatants were measured using a commercially available ELISA kit (R&D Systems, USA).

(3) Ultraviolet Irradiation Conditions

After discarding the medium, PBS(−) was added and UVB was irradiated at 30 or 60 mJ/cm². Following the irradiation, the PBS(−) was discarded, 2 ml of the medium or test solution dissolved in the medium was added, and the culture supernatants were collected after 24 hours.

(4) Preparation of RNA and cDNA

Keratinocytes were treated with the test solution and cells treated for 6 hours were used. Total RNA was extracted from the cells by addition of ISOGEN (Nippon Gene) according to the manufacturer's manual. cDNA was prepared with M-MLV reverse transcriptase (Life Technologies, Rockville USA), using 1 μg of RNA as template.

(5) Quantitation of Gene Expression by RT-PCR (Taqman-PCR) using Fluorescent Probes

The gene sequence of the obtained cDNA was determined quantitatively by Taqman-PCR using an ABI PRISM 7700 Sequence Detector (Perkin Elmer) according to the manufacturer's manual, in the manner described in paragraph 11 of Japanese Unexamined Patent Publication (Kokai) No. 11-32799. The result was normalized on the basis of the expression level of human GAPDH (glyceraldehyde-3-phosphate dehydrogenase) used as an internal standard.

(6) Sequences of Primers and Fluorescent Probes (Taqman Probes) for Taqman-PCR

| | |
|---|---|
| IL-6 | |
| forward primer | 5′ GAACTCCTTCTCCACAAGCG 3′ |
| IL-6 | |
| reverse primer | 5′ AGATGCCGTCGAGGATGTA 3′ |
| probe | |
| | 5′ TTCGTTCTGAAGAGGTGAGTGGCTG 3′ |
| IL-8 | |
| forward primer | 5′ TCAGAGACAGCAGAGCACACA 3′ |
| IL-8 | |
| reverse primer | 5′ CTCGGCAGCCTTCCTGATT 3′ |
| probe | |
| | 5′ AACATGACTTCCAAGCTGGCCA 3′ |
| GAPDH | |
| forward primer | 5′ GAAGGTGAAGGTCGGAGTC 3′ |
| GAPDH | |
| reverse primer | 5′ GAAGATGGTGATGGGATTTC 3′ |
| probe | |
| | 5′ AGGCTGAGAACGGGAAGCTTG 3′ |

(7) Assay of the Release of ATP in Ultraviolet Irradiated Keratinocytes

Ultraviolet rays were irradiated at an intensity of 10-200 mJ/cm² using UVB as the light source. After discarding the medium from the cell-cultured 12-well plate, PBS(−) was added and UVB was irradiated. Following irradiation, 10011 of cell supernatants were immediately sampled and the ATP in the supernatants were quantitated based on light emission using an ATP determination kit (Molecular Probes) according to the manufacturer's manual.

(8) Confirmation Test for Inflammation Suppressing Effect of Reactive Blue 2

After raising HR-1 mice for 48 hours in a dry environment (<10% RH), the dorsal skin was acetone-treated to disrupt its skin barrier function, and then the skin was applied with 1 mM Reactive blue 2. Control animals were applied with water. After an additional 48 hours, the dorsal skins of both animal groups were collected. The skin tissue was fixed with formaldehyde and subjected to hematoxylin-eosin (HE) staining and observed under an optical microscope.

### Experiment 1

(1) Assay of ATP-Induced IL-6 Release from Keratinocytes

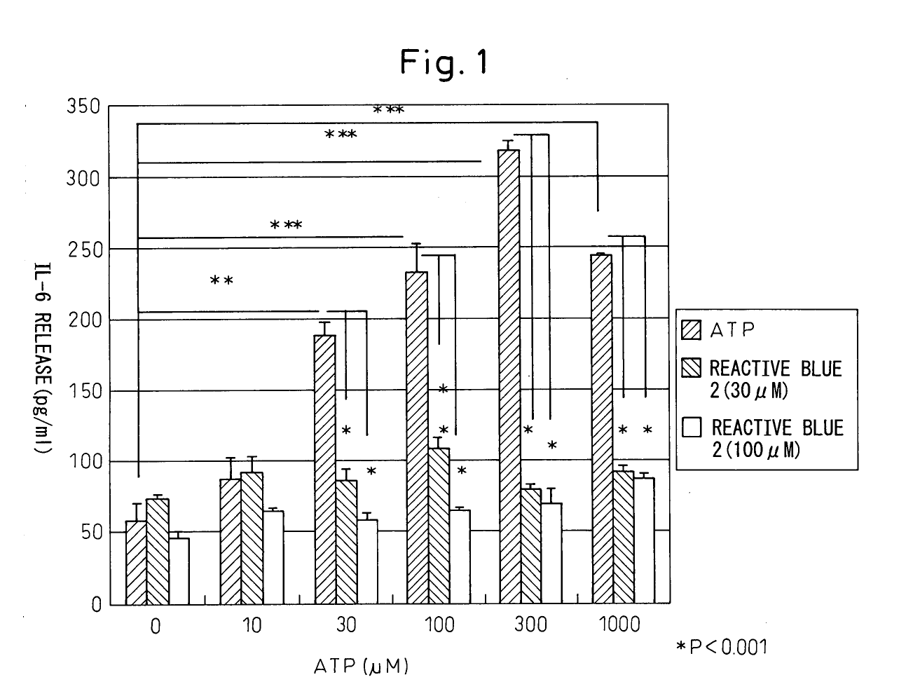

ATP (10 μM-1 mM) dissolved in the culture medium was applied as test solution to keratinocytes, and after 24 hours the culture supernatants were collected and the inflammatory cytokine IL-6 was assayed with the ELISA kit. The results are shown in FIG. 1.

As seen in FIG. 1, addition of ATP resulted in significant increase in IL-6 in proportion to the concentration of the added ATP. Further, when ATP was added 10 minutes after pretreatment of the keratinocytes with 30-100 μM Reactive blue 2 (ALEXIS, San Diego, Calif., USA), an antagonist for the ATP receptor subtype P2Y, the ATP-induced increase in IL-6 was significantly inhibited. These results are also shown in FIG. 1. The increase in IL-6 release produced by ATP addition was similarly inhibited by pretreatment of the cells with suramin, a known ATP receptor antagonist, as occurred with Reactive blue 2 (data not shown). This demonstrated that an ATP-induced increase in IL-6 release by keratinocytes is mediated by the ATP receptor subtype P2Y.

### Experiment 2

(2) Assay of ATP-Induced IL-8 Release from Keratinocytes

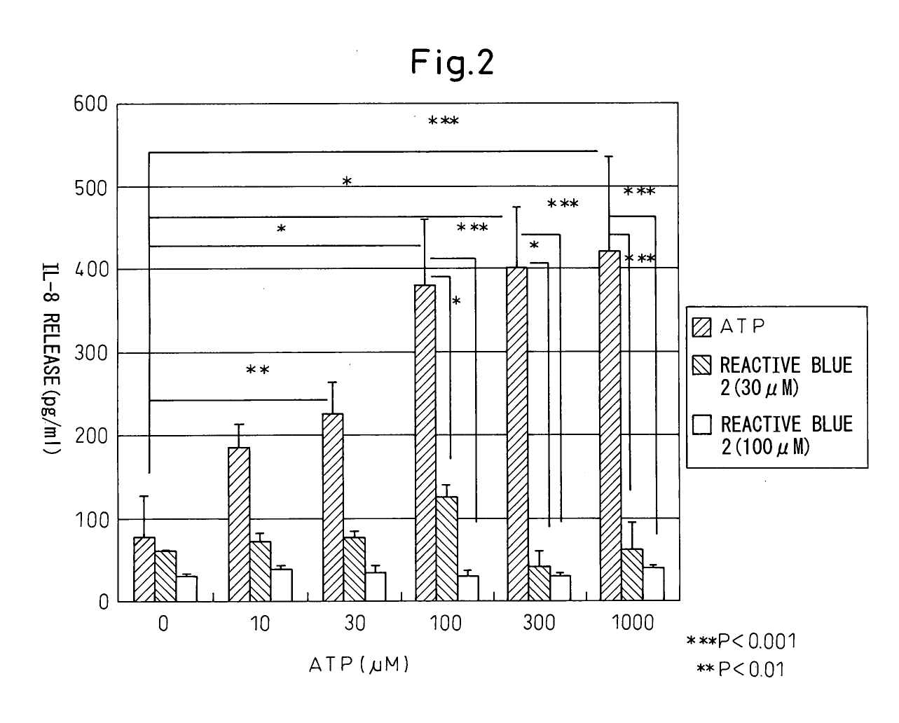

ATP (10 μM-1 mM) dissolved in the culture medium was applied as a test solution to keratinocytes, and after 24 hours the culture supernatants were recovered and the inflammatory cytokine IL-8 was assayed with an ELISA kit. The results are shown in FIG. 2.

As seen in FIG. 2, addition of ATP resulted in significant increase in IL-8 in proportion to the concentration of the added ATP. Further, when ATP was added 10 minutes after pretreatment of the keratinocytes with 30-100 μM Reactive blue 2, an antagonist for the ATP receptor subtype P2Y, the ATP-induced increase in IL-8 was significantly inhibited. These results are also shown in FIG. 2. Moreover, the increase in IL-8 release produced by ATP addition was similarly inhibited by pretreatment of the cells with suramin, a known ATP receptor antagonist, as occurred with Reactive blue 2 (data not shown). This demonstrated that ATP-induced increase in IL-8 release by keratinocytes is mediated by the ATP receptor subtype P2Y.

### Experiment 3

(3) Assay of ATP-Induced IL-1α Release from Keratinocytes

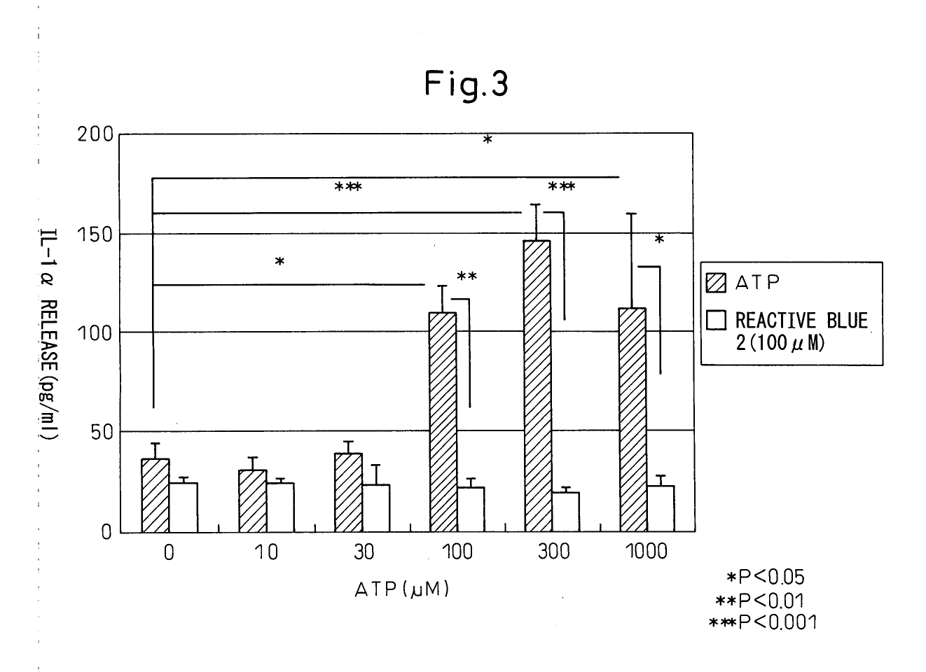

ATP (10 μM-1 mM) dissolved in the culture medium was applied as a test solution to keratinocytes, and after 24 hours the culture supernatants were collected and the inflammatory cytokine IL-1α was assayed with an ELISA kit. The results are shown in FIG. 3.

As seen in FIG. 3, addition of ATP resulted in significant increase in IL-1α in proportion to concentration. When ATP was added 10 minutes after pretreatment of the keratinocytes with 100 μM Reactive blue 2, an antagonist for the ATP receptor subtype P2Y, the ATP-induced increase in IL-1α was significantly inhibited. These results are also shown in FIG. 3. Moreover, the increase in IL-1α release produced by ATP addition was similarly inhibited by pretreatment of the cells with suramin, a known ATP receptor antagonist, as occurred with Reactive blue 2 (data not shown). This demonstrated that ATP-induced increase in IL-1α release by keratinocytes is mediated by the ATP receptor subtype P2Y.

### Experiment 4

(4) Assay of ATP-Induced TNFa Release from Keratinocytes

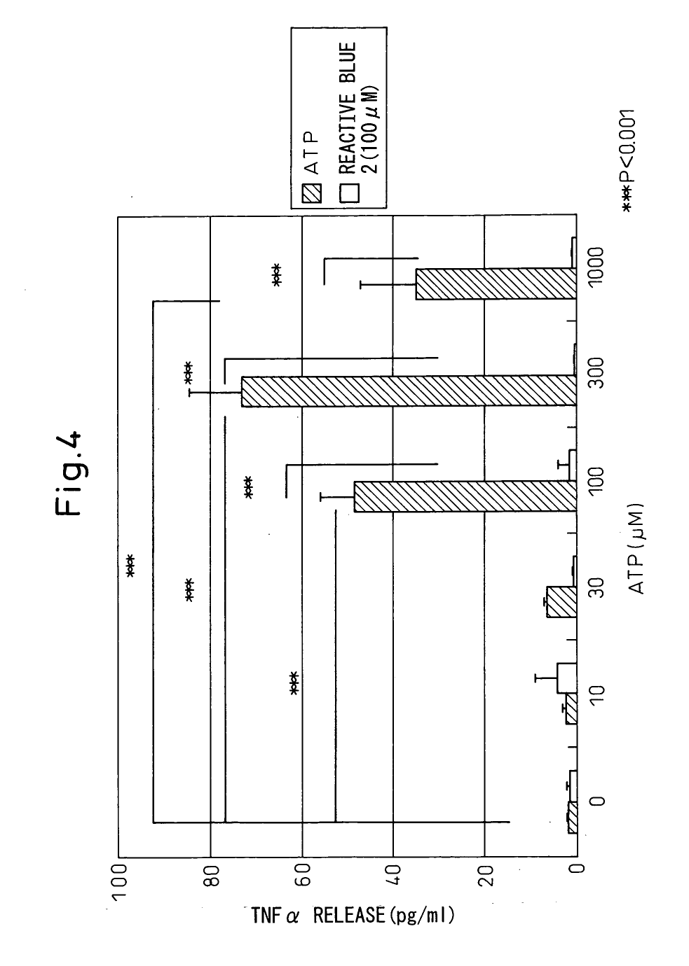

ATP (10 μM-1 mM) dissolved in the culture medium was applied as a test solution to keratinocytes, and after 24 hours the culture supernatants were recovered and the inflammatory cytokine TNFα was assayed with an ELISA kit. The results are shown in FIG. 4.

As seen in FIG. 4, addition of ATP resulted in significant increase in TNFa in proportion to concentration. When ATP was added 10 minutes after pretreatment of the keratinocytes with 100 μM Reactive blue 2, an antagonist for the ATP receptor subtype P2Y, the ATP-induced increase in TNFa was significantly inhibited. These results are also shown in FIG. 4. Moreover, the increase in TNFa release produced by ATP addition was similarly inhibited by pretreatment of the cells with suramin, a known ATP receptor antagonist, as occurred with Reactive blue 2 (data not shown). This demonstrated that ATP-induced increase in TNFα release by keratinocytes is mediated by the ATP receptor subtype P2Y.

### Experiment 5

(5) Effects of ATP and its Antagonist on IL-6 Gene Expression in Keratinocytes

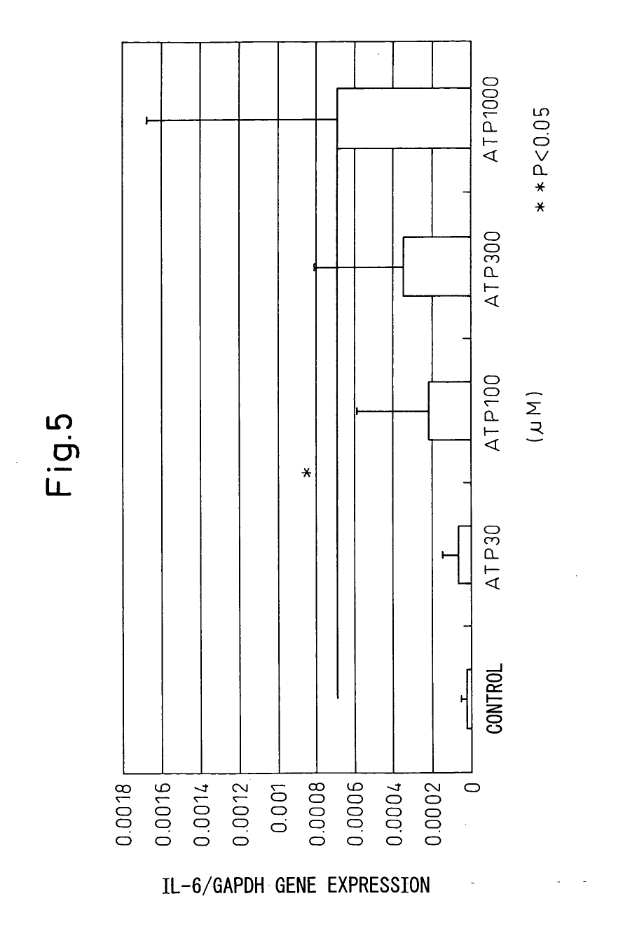

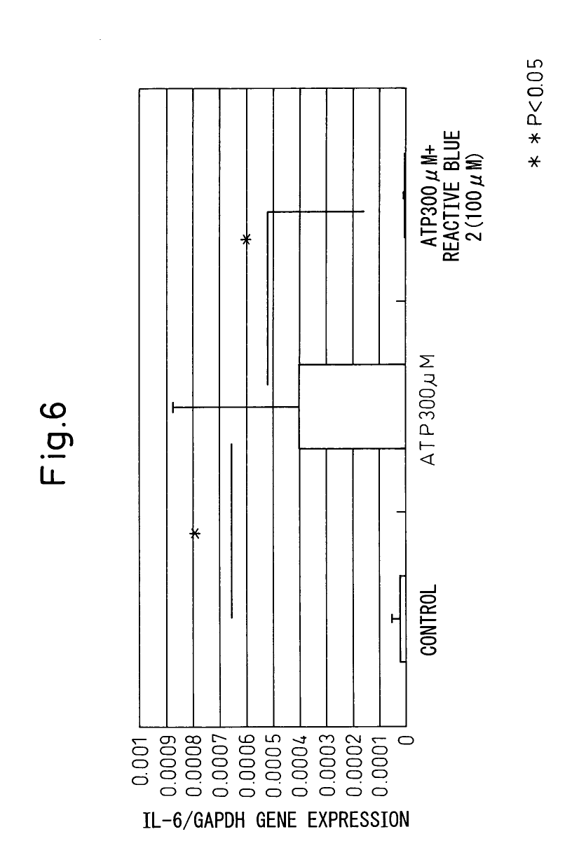

The keratinocytes treated with ATP in Experiment (1) were analyzed by RT-PCR (Taqman-PCR: Japanese Unexamined Patent Publication (Kokai) No. 11-32799) for quantitation of IL-6 gene expression and, as shown in FIG. 5, it was confirmed that ATP (30-1000 μM) significantly increased IL-6 expression in a concentration-dependent manner over non-stimulated cells (controls). Also, as shown in FIG. 6, it was confirmed by PCR that the ATP-induced increase in IL-6 gene expression was significantly inhibited by Reactive blue 2 (100 μM). The increase in IL-6 release produced by ATP addition was similarly inhibited by pretreatment of the cells with suramin, a known ATP receptor antagonist, as occurred with Reactive blue 2 (data not shown). This demonstrated that an ATP-induced increase in IL-6 release from keratinocytes is mediated by P2Y receptors.

### Experiment 6

(6) Effects of ATP and its Antagonist on IL-8 Gene Expression in Keratinocytes

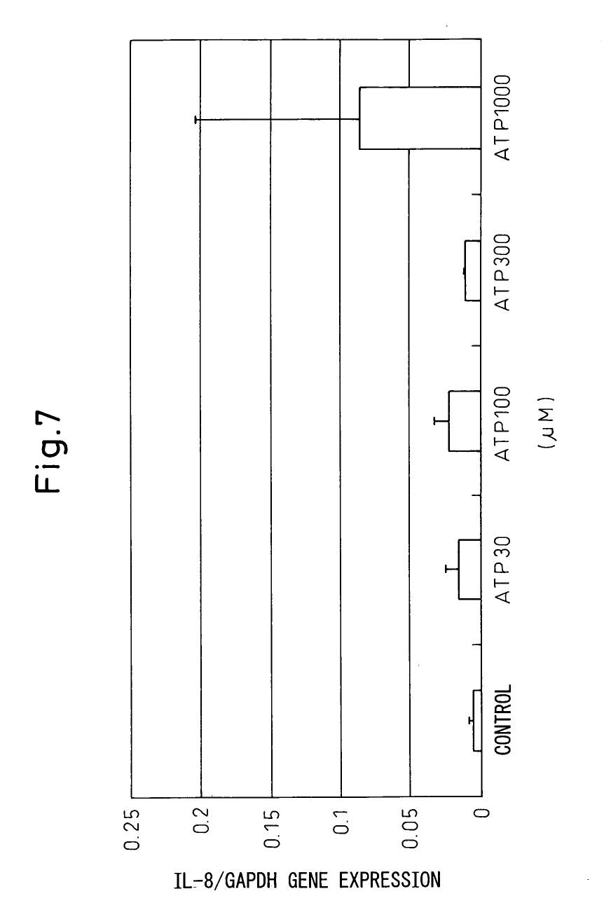

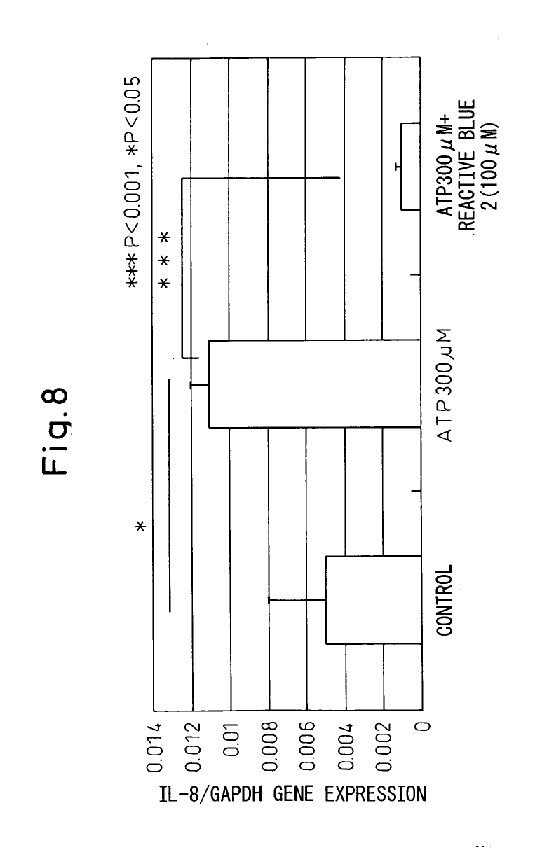

The keratinocytes treated with ATP in Experiment (2) were analyzed by RT-PCR (Taqman-PCR) for quantitation of IL-8 gene expression and, as shown in FIG. 7, it was confirmed that ATP (30-1000 μM) significantly increased IL-8 expression in a concentration-dependent manner over non-stimulated cells (controls). Also, as shown in FIG. 8, it was confirmed by PCR that the ATP-induced increase in IL-8 gene expression was significantly inhibited by Reactive blue 2 (100 μM). The increase in IL-8 release produced by ATP addition was similarly inhibited by pretreatment of the cells with suramin, a known ATP receptor antagonist, as occurred with Reactive blue 2 (data not shown). This demonstrated that ATP-induced increase in IL-8 release from keratinocytes is mediated by P2Y receptors.

### Experiment 7

(7) Assay of ATP Release Following Ultraviolet Irradiation

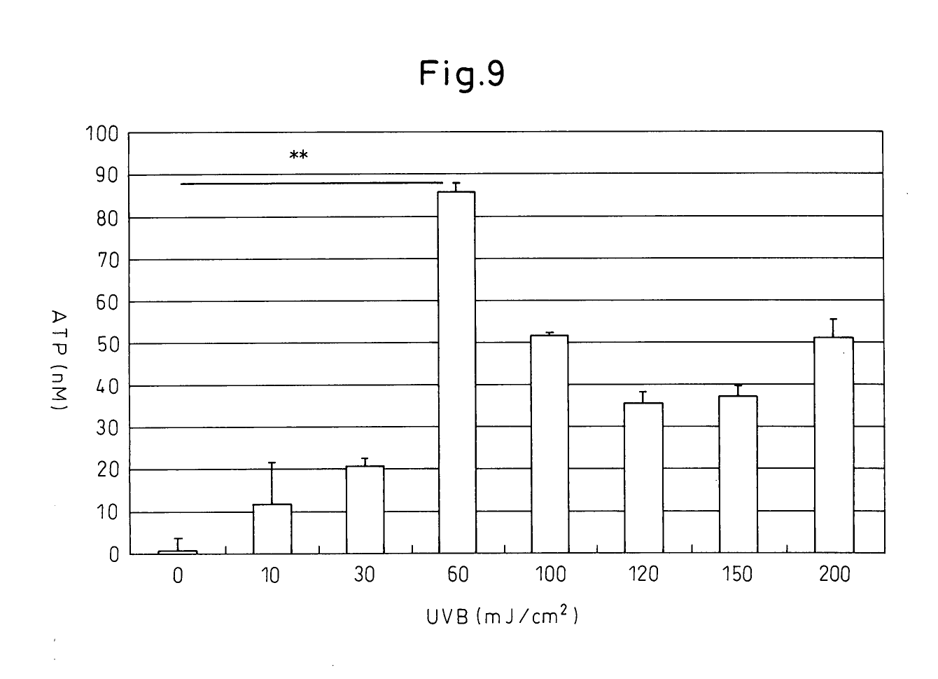

In order to confirm whether ATP is released into cell medium from keratinocytes by ultraviolet stimulation, ultraviolet irradiation with various intensities were applied to a medium. ATP release was found to peak at an ultraviolet intensity of 60 mJ/cm², as shown in FIG. 9. It was thus confirmed that exposure of cells to stimulation by ultraviolet rays results in release of ATP.

### Experiment 8

(8) Effect of ATP Antagonist on the Release of Cytokine IL-6 in Ultraviolet Irradiated Keratinocytes

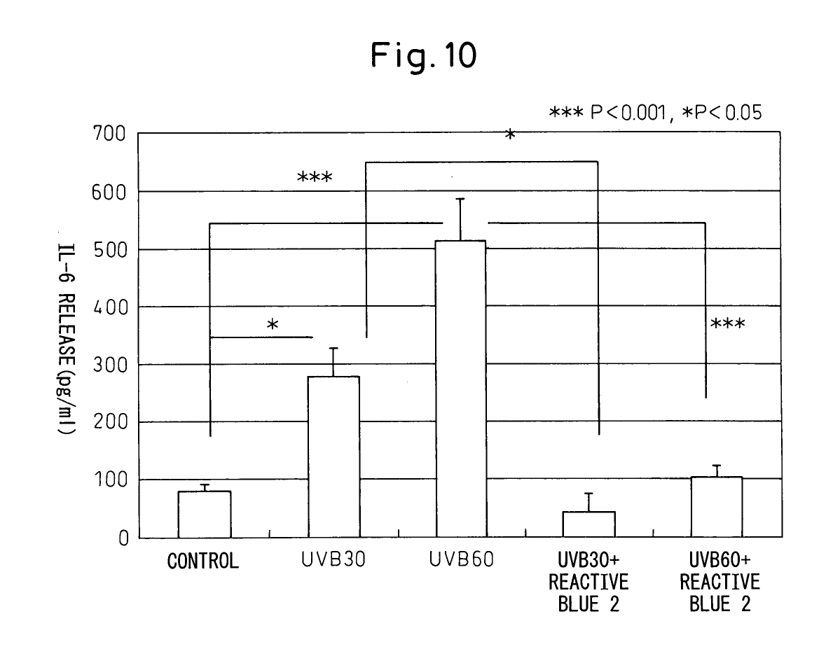

Cells were exposed to ultraviolet irradiation. After 24 h, keratinocyte cell supernatants were collected and the content of IL-6 was assayed with an ELISA kit. The results are shown in FIG. 10. As can be seen in FIG. 10, the amount of IL-6 increased significantly in proportion to ultraviolet irradiation intensity. Further, when the ultraviolet irradiation was carried out 10 minutes after pretreatment of the keratinocytes with 100 μM Reactive blue 2, an antagonist for the ATP receptor subtype P2Y, the ultraviolet irradiation-induced increase in IL-6 was significantly inhibited. These results are also shown in FIG. 10. Moreover, the increase in IL-6 release produced by ultraviolet irradiation was similarly inhibited by pretreatment of the cells with suramin, a known ATP receptor antagonist, as occurred with Reactive blue 2 (data not shown). This demonstrated that agonists for the ATP receptor subtype P2Y are effective against ultraviolet irradiation-induced increase in IL-6 release by keratinocytes.

### Experiment 9

(9) Effect of ATP Antagonist on the Release of Cytokine IL-8 in Ultraviolet Irradiated Keratinocytes

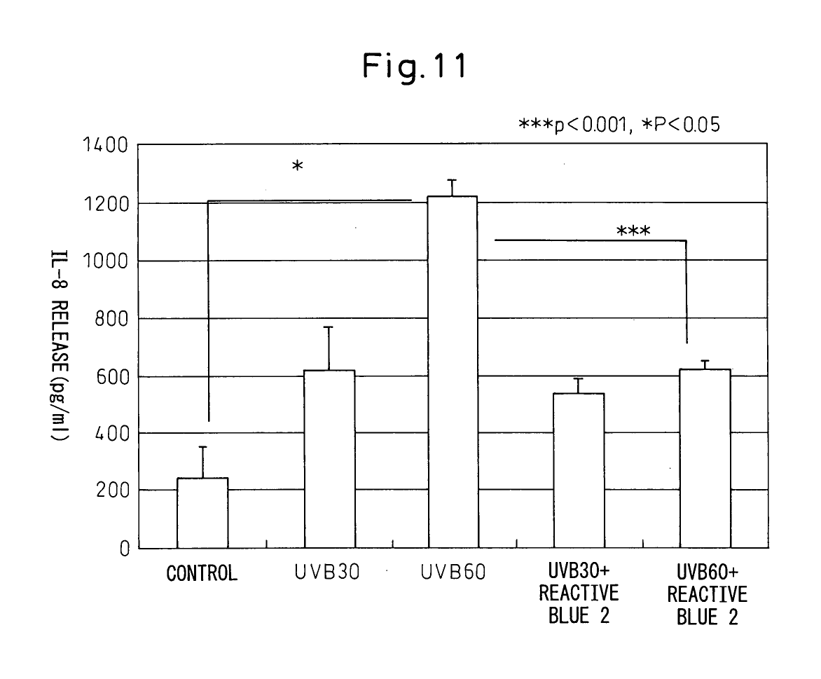

Cells were exposed to ultraviolet irradiation. After 24 h, keratinocyte cell supernatants were collected and the content of IL-8 was assayed with an ELISA kit. The results are shown in FIG. 11. As seen in FIG. 11, the amount of IL-8 increased significantly in proportion to ultraviolet irradiation intensity. Further, when the ultraviolet irradiation was carried out 10 minutes after pretreatment of the keratinocytes with 100 μM Reactive blue 2, an antagonist for the ATP receptor subtype P2Y, the ultraviolet irradiation-induced increase in IL-8 was significantly inhibited. These results are also shown in FIG. 11. Moreover, the increase in IL-8 release produced by ultraviolet irradiation was similarly inhibited by pretreatment of the cells with suramin, a known ATP receptor antagonist, as occurred with Reactive blue 2 (data not shown). This demonstrated that agonists for the ATP receptor subtype P2Y are effective against ultraviolet irradiation-induced increase in IL-8 release by keratinocytes.

### Experiment 10

(10) Inflammation Suppressing Effect of Reactive Blue 2

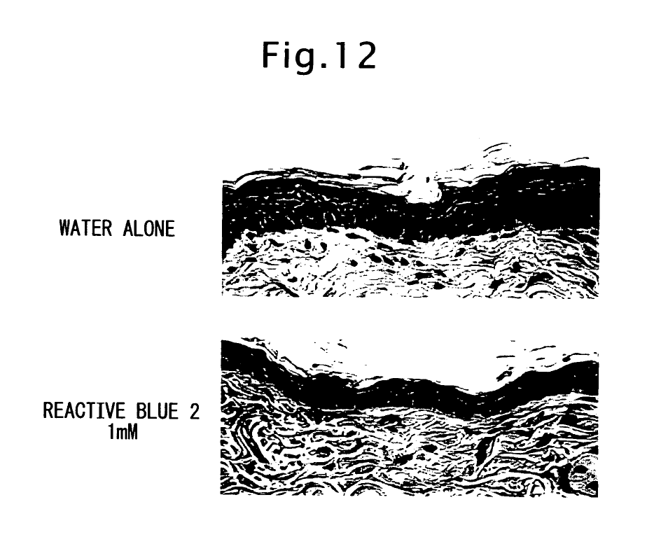

Barrier disruption by acetone treatment in a dry environment (<10% RH) was observed to accelerate epidermal hyperplasia of the epidermis based on HE staining, as seen in the upper panel of FIG. 12. The lower panel shows that coating with 1 mM Reactive blue 2 after barrier disruption inhibits the epidermal hyperplasia. It was thus demonstrated that a P2Y receptor antagonist is effective not only against ultraviolet stimulation but also against barrier disruption irritation occurring in a dry environment.

1. A method for suppression of inflammation, characterized by allowing an ATP receptor antagonist to act on ATP receptors of cells to block said receptors, thereby inhibiting release of an inflammatory cytokine by said cells.

2. The method according to claim 1, wherein said inflammatory cytokine is interleukin-6 (IL-6) and/or interleukin-8 (IL-8).

3. The method according to claim 1, wherein said inflammation is cutaneous inflammation induced by ultraviolet irradiation or dry irritation.

4. The method according to claim 1, wherein said cells are keratinocytes.

5. The method according to claim 1, wherein said ATP receptor antagonist is Reactive blue 2.

6. A pharmaceutical composition or cosmetic composition for suppression of inflammation, characterized by comprising an ATP receptor antagonist in an amount effective to act on ATP receptors of cells, thereby inhibiting release of an inflammatory cytokine by said cells.

7. A pharmaceutical composition or cosmetic composition according to claim 6, wherein said inflammatory cytokine is IL-6 and/or IL-8.

8. A pharmaceutical composition or cosmetic composition according to claim 6, wherein said inflammation is cutaneous inflammation induced by ultraviolet irradiation or dry irritation.

9. A pharmaceutical composition or cosmetic composition according to claim 6, wherein said cells are keratinocytes.

10. A pharmaceutical composition or cosmetic composition according to claim 6, wherein said ATP receptor antagonist is Reactive blue 2.

11. The method according to claim 2, wherein said inflammation is cutaneous inflammation induced by ultraviolet irradiation or dry irritation.

12. A pharmaceutical composition or cosmetic composition according to claim 7, wherein said inflammation is cutaneous inflammation induced by ultraviolet irradiation or dry irritation.


SEQUENCE LISTING (9 sequences)

SEQ ID NO: 1 - DNA, 20 bp, Artificial Sequence
  Forward Primer
gaactccttctccacaagcg

SEQ ID NO: 2 - DNA, 19 bp, Artificial Sequence
  Reverse Primer
agatgccgtcgaggatgta

SEQ ID NO: 3 - DNA, 25 bp, Artificial Sequence
  Probe
ttcgttctgaagaggtgagtggctg

SEQ ID NO: 4 - DNA, 21 bp, Artificial Sequence
  Forward Primer
tcagagacagcagagcacaca

SEQ ID NO: 5 - DNA, 19 bp, Artificial Sequence
  Reverse Primer
ctcggcagccttcctgatt

SEQ ID NO: 6 - DNA, 22 bp, Artificial Sequence
  Probe
aacatgacttccaagctggcca

SEQ ID NO: 7 - DNA, 19 bp, Artificial Sequence
  Forward Primer
gaaggtgaaggtcggagtc

SEQ ID NO: 8 - DNA, 20 bp, Artificial Sequence
  Reverse Primer
gaagatggtgatgggatttc

SEQ ID NO: 9 - DNA, 21 bp, Artificial Sequence
  Probe
aggctgagaacgggaagcttg